(19) United States
(12) Patent Application Publication
(10) Pub. No.: US 2011/0160688 A1
(43) Pub. Date: Jun. 30, 2011

(54) ABSORBENT ARTICLE COMPRISING LOTION COMPOSITION COMPRISING OMEGA-6 FATTY ACID AND METHOD OF IMPROVING SKIN BARRIER FUNCTION

(76) Inventor: Raphael WARREN, Amberley Village, OH (US)

(21) Appl. No.: 12/974,674

(22) Filed: Dec. 21, 2010

Related U.S. Application Data
(60) Provisional application No. 61/291,069, filed on Dec. 30, 2009.

(51) Int. Cl. A61L 15/44

(52) U.S. Cl. 604/367

(57) ABSTRACT

An absorbent article comprises a lotion composition comprising omega-6 fatty acid. A method of improving skin barrier function of vulvar skin comprising the step of contacting the vulvar skin with an absorbent article comprising a body facing surface and a garment facing surface, wherein omega-6 fatty acid is disposed on the body facing surface of the absorbent article.

### CROSS REFERENCE TO RELATED APPLICATION

This application claims the benefit of U.S. Provisional Application No. 61/291,069, filed Dec. 30, 2009.

### FIELD OF THE INVENTION

The present invention relates to an absorbent article comprising a lotion composition comprising omega-6 fatty acid and a method of improving skin barrier function of vulvar skin by contacting the vulvar skin with the absorbent article.

### BACKGROUND OF THE INVENTION

Disposable absorbent articles, such as diapers, training pants, and catamenial devices having lotioned topsheets are known. Lotions of various types are known to provide various skin benefits, such as prevention or treatment of diaper rash. These lotions can be applied to the topsheet of absorbent articles, for example, and can be transferred to the skin of the wearer during use.

The application of lotion compositions to topsheets of absorbent articles have been primarily directed to baby diapers, with the benefit provided being better skin health for the bottom of the baby. Little attention has been directed to the unique problems associated with the skin of an adult woman when wearing a feminine hygiene product. The skin of the vulvar area of an adult woman is very different than that of a baby's bottom (or buttock skin in general). For example, the vulvar area will generally be populated with hair. It is known that adult onset hormones (i.e., estrogens, progestins, corticosteroids) influence the disposition of the epidermis and dermis, the production of lubricating skin lipids, epidermal structural elements (i.e., kertains), or moisturizing factors. The vulvar skin is considerably thicker than other types of skin, with considerably more skin folds. Furthermore, hormonal changes associated with the onset of a woman's period can affect her skin sensitivity. These factors contribute to skin barrier function and to vulvar skin wellness in particular.

Independent of the menstrual cycle, vulvar skin also has an inferior skin barrier function and a high skin turnover rate comparable to those experiencing moderate skin lesions of psoriasis or those with atopic dermatitis, as compared to the reference standard, the volar forearm. Despite being a naturally humid environment (by virtue of wearing garments), a byproduct of wearing feminine hygiene products is the feeling of discomfort, skin chafing, and increased sensitivity. To compensate for these feelings and improve vulvar skin barrier function and skin wellness, women adapt habits such as frequent showering, frequent and costly catamenial pad changes, application of moisturizers and similar medicants. It would be desirable if an absorbent article in contact with the vulvar area, could attenuate this discomfort. It would be even more desirable if an absorbent article in contact with the vulvar area could improve vulvar skin wellness and skin barrier function by enabling greater resistance to environmental insults.

Accordingly there is a continuing desire for an absorbent article that reduces the discomfort associated with wearing absorbent articles and can improve the vulvar skin wellness and skin barrier function of vulvar skin.

### SUMMARY OF THE INVENTION

The present invention relates to an absorbent article comprising a lotion composition comprising omega-6 fatty acid. The absorbent article typically comprises a topsheet, a backsheet, an absorbent core disposed between the topsheet and the backsheet, and a lotion composition disposed on one or more layers of the absorbent article.

In one embodiment, the lotion composition comprises (a) an oil material comprising at least about 3%, by weight of the oil material, of omega-6 fatty acid, wherein the oil material has an oil stability index of at least about 10 hours, and (b) a carrier.

In another embodiment, the lotion composition comprises (a) at least about 0.00015%, by weight of the lotion composition, of omega-6 fatty acid, (b) at least about 0.0005%, by weight of the lotion composition, of oleic acid, and (c) a carrier.

The present invention further relates to a method of improving skin barrier function of vulvar skin, the method comprising contacting vulvar skin with an absorbent article comprising omega-6 fatty acid disposed on the body facing surface of the absorbent article.

### BRIEF DESCRIPTION OF THE DRAWING

The FIGURE is a top view of an absorbent article comprising a topsheet, backsheet, and an absorbent core, with a lotion composition applied thereto.

### DETAILED DESCRIPTION OF THE INVENTION

As used herein, the term “absorbent article” refers to devices that absorb and contain body exudates, primarily menses and/or urine. The term “disposable” is used herein to describe absorbent articles which are not intended to be laundered or otherwise restored or reused as an absorbent article after a single use. Examples of absorbent articles include feminine hygiene garments such as sanitary napkins, pantiliners, interlabial devices, hemorrhoid pads, wipes, tampons, and the like.

Disposable absorbent articles and components thereof, including the topsheet, backsheet, absorbent core, and any individual layers of these components, have a body surface and a garment surface. As used herein, “body surface” means that surface of the article or component which is intended to be worn toward or adjacent to the body of the wearer, while the “garment surface” is on the opposite side and is intended to be worn toward or placed adjacent to the wearer's undergarments when the disposable absorbent article is worn.

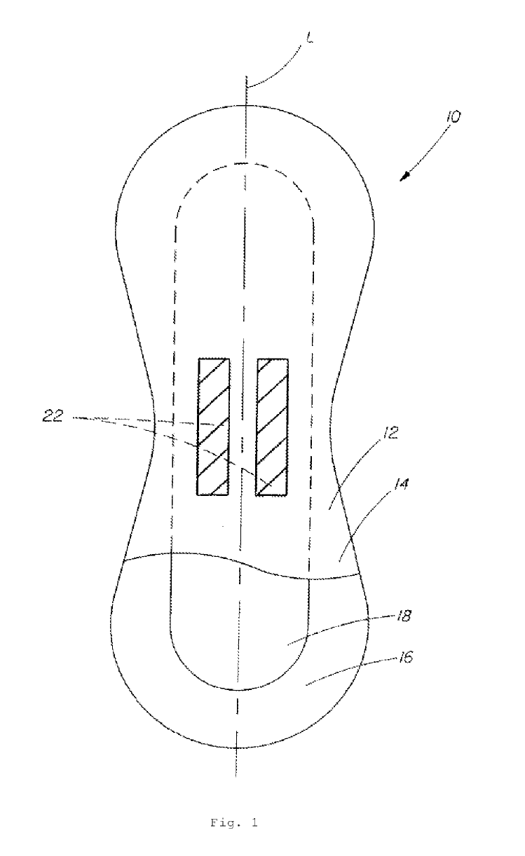

FIG. 1 shows an absorbent article 10, that can be a sanitary napkin or pantiliner, having a body facing surface 12 comprising a topsheet 14, a liquid impervious backsheet 16 joined to the topsheet 14, an absorbent core 18. The absorbent article 10 has a longitudinal axis L and may also be provided with additional features commonly found in napkins, including “wings” or “flaps” (not shown) as is known in the art and/or a fluid acquisition layer to promote fluid transport to the absorbent core 18. Likewise, the topsheet of the absorbent article can have various optional characteristics, as is known in the art. For example, the topsheet 14 can have channels embossed therein to direct fluid flow, and can have apertures therethrough to aid in fluid acquisition. The topsheet 14 of the absorbent article 10 of the present invention has a lotion composition 22 disposed onto the topsheet.

The topsheet is preferably compliant, soft feeling, and non-irritating to the wearers skin and hair. Further, the topsheet is liquid pervious, permitting liquids (e.g., menses and/or urine) to readily penetrate through its thickness. A suitable topsheet may be manufactured from a wide range of materials such as woven and nonwoven materials (e.g., a nonwoven web of fibers); polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the topsheet comprises a nonwoven web, the web may be manufactured by a wide number of known techniques. For example, the web may be spunbonded, carded, wet-laid, melt-blown, hydroentangled, combinations of the above, or the like.

The backsheet is impervious to liquids (e.g., menses and/or urine) and is preferably manufactured from a thin plastic film, although other flexible liquid impervious materials may also be used. As used herein, the term “flexible” refers to materials which are compliant and will readily conform to the general shape and contours of the human body. The backsheet prevents the exudates absorbed and contained in the absorbent core from wetting articles which contact the absorbent article such as bedsheets, pants, pajamas and undergarments. The backsheet may thus comprise a woven or nonwoven material, polymeric films such as thermoplastic films of polyethylene or polypropylene, or composite materials such as a film-coated nonwoven material. In one embodiment, the backsheet can be a breathable backsheet such as that described in U.S. Pat. No. 6,623,464.

The backsheet and the topsheet are positioned adjacent the garment surface and the body surface, respectively, of the absorbent core. The absorbent core can be joined with the topsheet, the backsheet, or both in any manner as is known by attachment means (not shown in FIG. 1) such as those well known in the art. However, embodiments of the present invention are envisioned wherein portions of the entire absorbent core are unattached to either the topsheet, the backsheet, or both.

In one embodiment, the topsheet of absorbent article 10 is made of a hydrophobic material. Therefore, if the topsheet is a nonwoven, the constituent fibers are preferably hydrophobic. Fibers are considered to be hydrophobic if film sheets formed from the polymers of the fibers would exhibit contact angles with water greater than 60 degrees, more preferably 75 degrees, and even more preferably greater than about 90 degrees. Contact angles as a measure of hydrophobicity are well known in the art, and methods for measuring contact angles are equally well known. As is well known, contact angles greater than about 90 degrees are considered hydrophobic, and contact angles less than 90 degrees are often considered hydrophilic. As used herein, however, contact angles of 60 degrees or greater are considered hydrophobic.

In another embodiment, the topsheet of absorbent article 10 is made of a hydrophilic material.

The use of absorbent articles, especially for feminine hygiene purposes, in the vulvar skin area can lead to various skin problems including irritation, chafing, and the like. Independent of the use of an absorbent article, it has been found that vulvar skin tends to exhibit deficiencies in omega-6 fatty acid content, especially in comparison to skin in other areas of the body, such as forearm skin. This deficiency in omega-6 fatty acid can result in inferior skin barrier and vulvar skin wellness. Therefore, increasing the omega-6 fatty acid content of vulvar skin can help to improve vulvar skin wellness and skin barrier function of vulvar skin. It can also reduce the potential for skin problems normally associated with the use of absorbent articles for feminine hygiene purposes.

To address this concern, the absorbent article of the present invention further comprises a lotion composition comprising omega-6 fatty acid. The lotion composition will typically comprise at least about 0.00015%, from about 0.00015% to about 10%, from about 0.0015% to about 7.5%, or from about 0.003% to about 5%, by weight of the lotion composition, of omega-6 fatty acid. Preferably, the omega-6 fatty acid of the lotion composition is esterified to the triacylglycerol component of an oil.

The omega-6 fatty acid will typically be contained in an oil material. Therefore, in one embodiment, the lotion composition comprises an oil material comprising omega-6 fatty acid. The lotion composition will typically comprise from about 0.005% to about 20%, from about 0.05% to about 15%, or from about 0.1% to about 10%, by weight of the lotion composition, of the oil material. The oil material will typically comprise at least 3%, from about 3% to about 50%, or from about 5% to about 40%, by weight of the oil material, of omega-6 fatty acid.

Unsaturated fatty acids, such as omega fatty acids, tend to be instable and tend to easily oxidize. Oxidation can be promoted by multiple sources that include temperature, light, air, oxygen, moisture, and metals. See, e.g., Belitz H-D, Grosch W, and Schieberle P, Lipids In Food Chemistry 3rd ed. Springer-Verlag, Heidelberg, 2004, p. 157-242. Indeed, common sources of product making can promote instability. For example, melting and mixing the lotion composition ingredients can require high temperatures (to a temperature above the melting point of the lotion composition ingredients, e.g., greater than 70° C.). In order to melt and preserve the uniformity of a semi-solid lotion composition, it is common to heat the lotion composition application tank to high temperatures (e.g., greater than 60° C., preferably above 70° C.) with mixing. Furthermore, the lotion composition can remain in the tank for a considerable amount of time (e.g., greater than 24 hours). Another source of instability can be the shelf storage of the finished product. It is not unusual for product to remain on the shelf (in the store or at home) for at least a year and, depending on geographical location, storage temperatures can exceed 40° C. Another source of instability can result from lotion compositions that are water- or glycol-based. Collectively, these factors can lead to oxidation and creation of reactive oxygen-free radicals or active oxygen. This can lead to product deterioration such as discoloration (i.e., yellowing) and/or rancid odor. When in contact with the skin, active oxygen can damage skin barrier function.

A common measure for monitoring oxidative stability is the development of hydroperoxides (peroxide value or PV) over time. Oxidative stability can also be expressed in terms of the time required to obtain secondary oxidation products when aerating a sample at elevated temperature. A suitable measure of oxidative stability is called the Oil Stability Index (referred to herein as “OSI”). The OSI of an oil material can be measured according to the American Oil Chemical Society Oil Stability Index Method (AOCS Official Method Cd 12b-92).

In one embodiment, the oil material of the present invention is selected to have an oil stability index (“OSI”) of at least about 10 hours, at least about 14 hours, or at least about 18 hours.

It is believed that oil materials comprising relatively high levels of oleic acid tend to be more stable in the context of the present invention. In one embodiment, the oil material of the present invention comprises at least about 10%, from about 10% to about 80%, or from about 15% to about 70%, by weight of the oil material, of oleic acid. In one embodiment, the lotion composition comprises from about 0.0005% to about 16%, from about 0.005% to about 12%, or from about 0.01% to about 8%, by weight of the lotion composition, of oleic acid.

It is believed that oil materials comprising relatively low levels of linolenic acid (omega-3 fatty acid) tend to be more stable in the context of the present invention. In one embodiment, the oil material of the present invention comprises less than about 10%, from about 10% to about 5%, or from about 5% to about 0%, by weight of the oil material, of linolenic acid. In one embodiment, the lotion composition comprises from about 2% to about 0%, from about 1% to about 0%, or from about 0.5% to about 0%, by weight of the lotion composition, of linolenic acid.

Non-limiting examples of suitable oil materials exhibiting the desired properties described herein include oleic canola Oil (Brassica campestris, B. napus, B. rapa; characterized by having an oleic content greater than 70%, e.g., hi oleic canola oil, very high oleic canola oil, or partially hydrogenated canola oil), marula kernel oil (Sclerocarya birrea), palm oil (Elaeis Guineensis Oil), palm olein, palm stearin, palm superolein, pecan oil, pumpkin seed oil, oleic safflower oil (Carthamus Tinctorius; characterized by having an oleic content of greater than about 30% and omega-6 fatty acid content of less than about 50%, e.g., hi oleic safflower oil), sesame oil (Sesamum indicum, S. oreintale), soybean oil (Glycine max, e.g., hi oleic soybean, low linolenic soybean oil, partially hydrogenated), oleic sunflower oil (Helianthus annus; characterized by having an oleic content of greater than about 40%, e.g., mid oleic sunflower or high oleic sunflower oil), and mixtures thereof. Oleic canola oil, palm oil, sesame oil, hi oleic safflower oil, hi oleic soybean oil, mid oleic sunflower oil, and high oleic sunflower oil are common plant-bred derived oils and may be also be derived from non-genetically modified organisms (non-GMO).

Non-limiting examples of oil materials are commercially-available from a number of vendors, including Cargill for partially hydrogenated soybean oil (i.e., Preference® 110W Soybean Oil or Preference® 300 Hi Stability Soybean Oil), mid oleic sunflower oil (i.e., NuSun® Mid-Oleic Sunflower Oil), high oleic sunflower oil (i.e., Clear Valley® High Oleic Sunflower Oil), high oleic canola oil, very high oleic canola, and partially hydrogenated low erucic rapeseed oil (i.e., Clear Valley® 65 High Oleic Canola Oil and Clear Valley® 75 High Oleic Canola Oil); Lambert Technology for high oleic canola oil (i.e., Oleocal C104); Arch Personal Care for marula kernel oil; Pioneer for high oleic soybean oil (i.e., Plenish®); Asoyia for low linolenic soybean oil (i.e., Ultra Low Linolenic Soybean Oil®); and Dipasa, Inc. for refined sesame oil.

It should be noted that the grade of oil material can be important as well in achieving the desired properties of the oil material as described herein. For example, the source of the oil material can be important, as the same oil (e.g. sesame oil) can exhibit a wide range of OSI values depending upon the source of the oil material.

The oil material can further comprise a blend of oils, including those described supra, as well as additional oil materials. Suitable additional oil materials can include acai berry oil, almond oil, avocado oil, beech oil, brazil nut oil, camelina sativa oil (family Brassicaceae, e.g. Camelina Sativa, Gold of Pleasure, False Flax, etc.), camellia seed oil, canola oil, carrot seed oil, cashew nut oil, caster oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaea), macadamia oil, maringa oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The oil material of the present invention can be selected from the group consisting of camelina sativa seed oil, oleic canola oil, evening primrose oil, manila kernel oil, palm oil, palm olein, palm stearin, palm superolein, passiflora incarnata seed oil, pecan oil, pumpkin seed oil, oleic safflower oil, sesame oil, soybean oil, oleic sunflower oil, vegetable oil and mixtures thereof.

Preferred oil materials of the present invention include a mixture of vegetable oil and camelina sativa seed oil (commercially-available as Lipex® Omega 3/6 from Aarhus Karlshamn Sweden AB), a mixture of vegetable oil and passiflora incarnata seed oil (commercially-available as Lipex® Omega Passiflora from Aarhus Karlshamn Sweden AB), a mixture of vegetable oil and evening primrose oil (commercially-available as Lipex Omega EPO from Aarhus Karlshamn Sweden AB), high oleic canola oil (commercially-available as Clear Valley® 75 High Oleic Canola Oil from Cargill), or mixtures thereof.

To further enhance the stability of the oil material, certain antioxidants can be added to certain oil materials or to the lotion composition. In one embodiment, the oil material comprises from about 0.005% to about 1%, from about 0.01% to about 0.5%, or from about 0.02% to about 0.2%, by weight of the oil material, of an antioxidant. In one embodiment, the lotion composition comprises from about 0.0005% to about 1%, from about 0.001% to about 0.75%, or from about 0.002% to about 0.5%, by weight of the lotion composition, of an antioxidant. Attempts have been made to stabilize oxidatively unstable oils with antioxidants with unpredictable outcomes. See, e.g., Merrill L I, Pike O A, Ogden L V, Oxidative stability of conventional and high-oleic vegetable oils with added anti-oxidants, J Am Oil Chem Soc 85:771-776, 2008; Chu Y-H and Hsu H-F, Effect of antioxidants on peanut oil stability, Food Chemistry 66:29-34, 1999; and Isbell T A, Abbott T P, and Carlson K D, Oxidative stability index of vegetable oils in binary mixture with meadowfoam oil, Ind Crops Products 9:115-123, 1998. Other antioxidants, such as the phenolic tert-butylhydroquinone (TBHQ), butylated hydroxytoluene (BHT), or butylated hydroxyanisole (BHA) have been reported to stabilize oils although these are known skin sensitizers and would have limited value in an absorbent product having direct contact with the skin. Furthermore, blending unstable and stable oils does not necessarily lead to acceptable oil stability profiles, however, and an undesirable consequence can be the dilution of the desirable omega-6 fatty acid below a level that is desirable.

Non-limiting examples of suitable antioxidants include α-tocopherol, β-tocopherol, γ-tocopherol, δ-tocopherol, tocotrienol, rosemary, sesamol, sesamolin, sesamin, catechin, and mixtures thereof.

The lotion composition of the present invention further comprises a carrier. The carrier can help to deliver the omega-6 fatty acid of the present invention to the skin of the wearer of the absorbent article. The carrier can be included in the compositions as an individual carrier or a combination of carrier ingredients. The carrier can be a liquid, solid, or semisolid carrier material, or a combination of these materials, and preferably forms a homogenous mixture or solution at selected processing temperatures for the resultant carrier system and at processing temperatures for combining the carrier with the cooling agents in formulating the lotion compositions herein. Processing temperatures for the carrier system typically range from about 60° C. to about 90° C., more typically from about 70° C. to about 85° C., even more typically from about 65° C. to about 80° C.

The lotion compositions of the present invention can comprise the carrier at a total carrier concentration ranging from about 60% to about 99.9%, preferably from about 70% to about 98%, more preferably from about 80% to about 97%, by weight of the lotion composition. Suitable carrier compounds include petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin and its derivatives, glyceride and its derivatives including acetoglycerides and ethoxylated glycerides of C12-C28 fatty acids, and mixtures thereof. Alternatively or in combination with, the carrier may also be composed of polysiloxane compounds non-limiting examples include dimethicones (1-100,000,000 centistoke), cyclomethicones, alkylated silicones (hair conditioning agents), silicone gums, silicone gels, silicone waxes, copolymers of silicone (vinyl dimethicone polymers, phenyl vinyl dimethicone polymers, alkylated silicone polymers, polyethylene oxide/silicone copolymers, polyethylene oxide/alkyl silicone copolymers), and mixtures thereof.

Nonlimiting examples of suitable petroleum-based hydrocarbons having from about 4 to about 32 carbon atoms include mineral oil, petrolatum, isoparaffins, various other branched chained hydrocarbons, and combinations thereof. Mineral oil is also known as “liquid petrolatum”, and usually refers to less viscous mixtures of hydrocarbons having from about 16 to about 20 carbon atoms. Petrolatum is also known as “mineral wax”, “petroleum jelly”, and “mineral jelly”, and usually refers to more viscous mixtures of hydrocarbons having from about 16 to about 32 carbon atoms. An example of commercially available petrolatum include petrolatum sold as Protopet® 1s which is available from the Sonneborn Corporation located in Mahwah, N.J.

Other carriers suitable herein can include oils or fats such as natural oils or fats, or natural oil or fat derivatives, in particular of plant or animal origin. Non-limiting examples include apricot oil, babassu oil, castor oil, coconut oil, cod liver oil, hydrogenated corn oil, hydrogenated cottonseed oil, hazelnut oil, jojoba oil, macadamia oil, meadowfoam seed oil, mink oil, maringa oil, manila oil, mortierella oil, palm kernel oil, hydrogenated peanut oil, hydrogenated rapeseed oil, rose hip oil, hydrogenated safflower oil, hydrogenated soybean oil, hydrogenated sunflower oil, hydrogenated walnut oil, hydrogenated wheat germ oil, or the hardened derivatives thereof Other non-limiting examples of fats and oils suitable as carriers herein include: butter, C12-C18 acid triglyceride, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, hydrogenated castor oil, hydrogenated castor oil laurate, hydrogenated coconut oil, hydrogenated C12-C18 triglycerides, hydrogenated fish oil, hydrogenated lard, hydrogenated menhaden oil, hydrogenated mink oil, hydrogenated orange roughy oil, hydrogenated shark liver oil, hydrogenated tallow, hydrogenated vegetable oil, lanolin and lanolin derivatives, lanolin alcohol, lard, lauric/palmitic/oleic triglyceride, lesquerella oil, maleated soybean oil, neatsfoot oil, oleic/linoleic triglyceride, oleic/palmitic/lauric/myristic/linoleic triglyceride, oleostearine, olive husk oil, omental lipids, pengawar djambi oil, pentadesma butter, phospholipids, shea butter, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, and the like, as well as mixtures thereof.

Other suitable carriers include mono- or di-glycerides, such as those derived from saturated or unsaturated, linear or branch chained, substituted or unsubstituted fatty acids or fatty acid mixtures. Examples of mono- or diglycerides include mono- or di-C₁₂₋₂₄fatty acid glycerides, specifically mono- or di-C₁₆₋₂₀fatty acid glycerides, for example glyceryl monostearate, glyceryl distearate.

Carriers can also include esters of linear C₆-C₂₂-fatty acids with branched alcohols.

The carrier of the present invention can also include sterols, phytosterols, and sterol derivatives as described in U.S. Pat. No. 6,534,074B.

Nonlimiting examples of suitable fatty alcohols having from about 12 to about 24 carbon atoms include saturated, unsubstituted, monohydric alcohols or combinations thereof, which have a melting point less than about 110° C., preferably from about 45° C. to about 110° C. Specific examples of fatty alcohol carriers for use in the lotion compositions of the present invention include, but are not limited to, stearyl alcohol, cetearyl alcohol, behenyl alcohol, arachidyl alcohol, lignocaryl alcohol, and combinations thereof. Examples of commercially available cetearyl alcohol is Stenol 1822 and behenyl alcohol is Lanette 22, both of which are available from the Cognis Corporation located in Cincinnati, Ohio.

Nonlimiting examples of suitable fatty acid esters include those fatty acid esters derived from a mixture of C₁₂-C₂₈ fatty acids and short chain (C₁-C₈, preferably C₁-C₃) monohydric alcohols preferably from a mixture of C₁₆-C₂₄ saturated fatty acids and short chain (C₁-C₈, preferably C₁-C₃) monohydric alcohols. Suitable fatty acid esters can also be derived from esters of longer chain fatty alcohols (C₁₂-C₂₈, preferably C₁₂-C₁₆) and shorter chain fatty acids such as lactic acid, specific examples of which include lauryl lactate and cetyl lactate. Representative examples of suitable fatty acid esters include methyl palmitate, methyl stearate, isopropyl laurate, isopropyl myristate, isopropyl palmitate, ethylhexyl palmitate, stearyl stearate, palmityl stearate, stearyl behenate, cetyl stearate, cetyl behenate, cetyl palmitate, cetearyl behenate, behenyl behenate, stearyl heptanoate, stearyl octanoate, myristyl myristate, myristyl isostearate, myristyl oleate, cetyl isostearate, cetyl oleate, stearyl isostearate, stearyl oleate, isostearyl myristat, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl isostearate, behenyl oleate, erucyl isostearate, and mixtures thereof.

Nonlimiting examples of suitable alkyl ethoxylates include C₁₂-C₂₂ fatty alcohol ethoxylates having an average degree of ethoxylation of from about 2 to about 30. Nonlimiting examples of suitable lower alcohols having from about 1 to about 6 carbon atoms include isopropanol, butanediol, 1,2,4-butanetriol, 1,2 hexanediol, ether propanol, and mixtures thereof. Nonlimiting examples of suitable low molecular weight glycols and polyols include ethylene glycol, polyethylene glycol (e.g., Molecular Weight 200-600 g/mole), butylene glycol, propylene glycol, polypropylene glycol and mixtures thereof. A more detailed description of carrier ingredients including suitable hydrocarbons, polysiloxane compounds, and fatty alcohol ethoxylates can be found in U.S. Pat. No. 5,643,588, issued Jul. 1, 1997 to Roe et al. entitled “Diaper Having A Lotioned Topsheet”.

Suitable carriers further encompass waxes. As used herein, the term ‘wax’ refers to oil soluble materials that have a waxy constituency and have a melting point or range of above ambient temperature, in particular above 25° C. Waxes are materials that have a solid to semi-solid (creamy) consistency, crystalline or not, being of relative low viscosity a little above their liquefying point. Suitable waxes which can be incorporated into the lotion composition include animal, vegetable, mineral or silicone based waxes which may be natural or synthetic, and including mixtures thereof. Waxes can include but are not limited to: natural waxes from vegetal origin, such as bayberry wax, beeswax, candelilla wax, carnauba, ceresin, shea butter, cocoa butter, Japan wax, jojoba wax, lanolin wax, ouricury wax, mink wax, montan wax, rice bran wax, steryl dimethicone, fruit-derived waxes, such as orange wax, lemon wax, and the like; and waxes from animal origin such as beeswax, woolwax, bear fat, and the like. Natural waxes further comprise mineral waxes such as ceresin and ozokerite waxes. Synthetic waxes comprise petroleum-based waxes, such as certain carrier materials described hereinbefore, such as paraffin, vaseline, petrolatum, micro wax, and microcrystalline wax. Further suitable synthetic waxes are polyalkylene and polyethyleneglycol waxes, e.g. polyethylene wax; waxes based on chlorinated naphtalenes such as ‘Halowax’, synthetic hydrocarbon waxes, and the like, PEG-6 beeswax, PEG-8 beeswax, C30 alkyl dimethicone, synthetic beeswax, synthetic candelilla wax, synthetic carnuba wax, synthetic japan wax, synthetic jojoba wax, motan acid wax, motan wax, ouricury wax, rezowax, including mixtures thereof.

Other wax components can be certain fats (including mono-, di- and triglycerides and fatty acid alkylesters), fatty alcohols, fatty acids, including substituted fatty acids (in particular hydroxy substituted fatty acids, for example, 12-hydroxystearic acid), dialkyl(ene)ethers, dialkyl(ene) carbonates, dicarboxylic acids (in particular the C₁₆-C₄₀-dialkylesters of dicarboxylic acids, e.g. the C₁₆-C₄₀-alkyl stearates, C₁₈-C₃₈-alkylhydroxystearyl stearates or C₂₀-C₄₀-alkyl erucates) and hydroxy fatty alcohols. Still further wax components are selected from the group of aromatic carbonic acids, tricarboxylic acids, or from the group of lactides of long-chained hydroxycarbonic acids. Myristyl lactate is a suitable carrier. Further wax components that can be used are C₃₀-C₅₀ alkyl bees wax; tri-C₁₆-C₄₀-alkyl citrates, e.g. tristearyl citrate, triisostearyl citrate, trilauryl citrate; ethyleneglycol di fatty acid esters, in particular the ethylene glycol di-C₁₂-C₃₀-fatty acid esters, e.g. ethylene glycol dipalmitate, ethyleneglycol distearate, and ethyleneglycol di(12-hydroxystearate).

Other suitable carriers include materials that act as solidifying agents, including some of the materials described hereinbefore. Suitable solidifying agent(s) in the lotion compositions of the present invention can function to help solidify the composition so that the composition is a solid at room temperature and has a melting point of at least 32° C. The solidifying agent may also provide a tackiness to the composition that improves the transfer by adhesion to the skin of the wearer. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer and combinations thereof. The solidifying agents can be selected from alkyl siloxanes, polymers, hydrogenated vegetable oils having a melting point of 35° C. or greater, fatty acid esters with a melting point of 35° C. or greater, alkyl hydroxystearates, branched esters, alkoxylated alcohols and alkoxylated carboxylic acid. Additionally, the solidifying agents can be selected from animal, vegetable and mineral waxes and alkyl silicones. Examples of suitable solidifying agents include, but are not limited to, the following: alkyl silicones, alkyl trimethylsilanes, beeswax, behenyl behenate, behenyl benzoate, C24-C28 alkyl dimethicone, C30 alkyl dimethicone, cetyl methicone, stearyl methicone, cetyl dimethicone, stearyl dimethicone, cerotyl dimethicone, candelilla wax, carnuba, synthetic carnuba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C20-C40 alkyl behenate, C2-C5 lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di C2-C5 alkyl fumerate, dibehenyl fumerate, myristyl lactate, myristyl lignocerate, myristyl myristate, myristyl stearate, lauryl stearate, octyidodecyl stearate; octyidodecyl stearoyl stearate, oleyl arachidate, oleyl stearate, tridecyl behenate, tridecyl stearate, tridecyl stearoyl stearate, pentaerythrityl tetrabehenate, penteerythritylhydrogenated rosinate, pentaerythrityl distearate, pentaerythrityltetraabeite, penteerythrityl tetracocoate, penteerythrityl tetraperlargonate, pentserythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C30-C60 fatty alcohols, C20+fatty amides, polypropylene, polystyrene, polybutane, polybutylene terephthalate, polydipentane, polypropylene, zinc stearate, dodecyl laurate, stearyl palmitate, octadecyl hexedecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C20-C40 alkyl hydroxystearyl stearate, and mixtures of such compounds.

The absorbent article of the present invention can optionally further comprise essential oil materials that help to connote the benefits provided by the absorbent article. Such essential oil materials can be incorporated into the absorbent article separate from the lotion composition or can be made part of the lotion composition. Non-limiting examples of suitable essential oil materials include Acorns gramineus, Anthemis nobilis, Artemisia dracunculus, Basil, Bergamot, Calamintha sylvatica, Caraway, Cedarwood, Chamomile, Cineol, Cinnamon, Cinnamon bark, Citrus aurantium, Clove, Cypress, Dill, Eucalyptus, Eugenol, Frankincense, Galangol, Geranium, Ginger, Hibiscus, Hop, Jasmine, Juniper, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Tea Tree, Thyme, Valerian, Ylang ylang, Zadoary, Hibiscus, or mixtures thereof. Preferred essential oils associated with arousal include Cypress, Hibiscus, Juniper, Cineol, Citrus, Sweet Orange, and Rosemary. Preferred oils associated with a harmonizing effect include Lavender, Neroli, and Ylang ylang.

The particular essential oils herein, such as described above, can be blended in a carrier at a concentration ranging from about 0.0001% to about 10.0%, from about 0.0001% to about 3.0%, from about 0.0001% to about 0.1%, from about 0.001% to about 1%, or from about 0.01% to about 1.0%, by weight of the lotion composition. The essential oil can also be prepared in a premix in an oil material herein. Nonetheless, the final concentration of the essential oil will typically fall in the ranges described above.

The absorbent article or lotion composition of the present invention can further comprise supplemental skin treatment agents such as niacinamide, zinc oxide, hexamidine, panthenol, and the like, and mixtures thereof. Suitable skin treatment agents are described in US 2003/0082219 A1.

The absorbent article or lotion composition of the present invention can further comprise a cooling agent. Suitable cooling agents are described in US 2004/0081680 A1 and US 2009/0240223 A1.

When applied to the outer surface of sanitary napkin topsheets, the lotion compositions of the present invention can be transferable to the wearer's skin by normal contact, wearer motion, and/or body heat, thereby providing omega-6 fatty acid to the skin of the wearer.

The sanitary napkin topsheets of the present invention contain an effective amount of the lotion composition. As used herein, the term “effective amount of a lotion composition” refers to an amount of a particular lotion composition which, when applied to a sanitary napkin topsheet, will be effective in transferring omega-6 fatty acid to the skin of the wearer. The effective amount of a lotion composition will depend, to a large extent, on the particular lotion composition used.

In preparing lotioned absorbent articles according to the present invention, the lotion composition can be applied to the outer surface (i.e., body facing surface) of the topsheet, but can also be applied to the inner surface of the topsheet or to any other component of the absorbent article. Any of a variety of application methods that evenly distribute the lotion composition can be used. Suitable methods include spraying, printing (e.g., flexographic printing), coating (e.g., gravure coating), extrusion, or combinations of these application techniques, e.g. spraying the lotion composition on a rotating surface, such as a calendar roll, that then transfers the composition to the outer surface of the topsheet.

The manner of applying the lotion composition to the topsheet, or other component, can be such that the topsheet does not become saturated with the lotion composition. If the topsheet becomes saturated with the lotion composition, there is a greater potential for the lotion to block the topsheet openings, reducing the ability of the topsheet to transmit fluid to the underlying absorbent core. Also, saturation of the topsheet is not required to obtain the therapeutic and/or protective lotion benefits. Particularly suitable application methods will apply the lotion composition primarily to the outer surface of the topsheet.

Generally, a safe and effective amount of the lotion composition is applied to a topsheet of an absorbent article described herein wherein such safe and effective amounts include applying from about 0.05 g/m² (0.032 mg/in²) to about 100 g/m² (64.5 mg/in²) preferably from about 0.5 g/m² (0.32 mg/in²) to about 50 g/m² (32.2 mg/in²), more preferably 1.0 g/m² (0.645 mg/in²) to about 30 g/m² (19.3 mg/in²) of the lotion composition to the topsheet of the absorbent article.

The lotion composition may be applied to the entire surface of the topsheet or portions thereof. The lotion composition can be applied in a stripe aligned with and centered on the longitudinal centerline of the disposable absorbent article. The lotion composition can be applied in a plurality of stripes having uniform or non-uniform widths. Alternatively the lotion can be aligned with and centered in apposition to the longitudinal centerline.

The lotion composition can also be applied nonuniformly to the outer surface of the sanitary napkin topsheet. By “nonuniform” is meant that the amount, pattern of distribution, etc. of the lotion composition can vary over the topsheet surface. For example, some portions of the treated surface of the topsheet can have greater or lesser amounts of lotion composition, including portions of the surface that do not have any lotion composition on it. For example, the lotion composition can be applied on one region of the topsheet in the shape of a rectangle and/or a circle, and/or as mutliplicity of dots.

The lotion composition can be applied to the topsheet or other component at any point during assembly. For example, the lotion composition can be applied to the topsheet of the finished disposable absorbent product before it has been packaged. The lotion composition can also be applied to the topsheet before it is combined with the other raw materials to form a finished disposable absorbent product.

The lotion composition is typically applied from a melt thereof to the absorbent article. Since the lotion composition will typically melt at significantly above ambient temperatures, it is usually applied as a heated coating. Typically, the lotion composition is heated to a temperature in the range from about 35° C. to about 100° C., preferably from 40° C. to about 90° C., prior to being applied. Once the melted lotion composition has been applied, it is allowed to cool and solidify to form solidified coating or film on the surface of the topsheet or other component. Preferably, the application process is designed to aid in the cooling/set up of the lotion.

Lotion compositions of the present invention can be applied by printing methods, or continuous spray or extrusion as is known in the art, or as is described in U.S. Pat. No. 5,968,025.

It can be preferred that the lotion be applied in a plurality of stripes parallel to the longitudinal axis of the absorbent article. This allows for both transfer of the lotion to a broader area of the vulva and improved fluid handling of the absorbent article.

In another embodiment, instead of (or in addition to) being applied to the topsheet of an absorbent article, the lotion composition can be applied to a wipe article that is supplied with the absorbent article (for example, as described in detail in U.S. Pat. No. 5,569,230, U.S. Pat. No. 6,911,022 or WO 03/057122 A1). In another embodiment, the lotion composition can be provided as a stand-alone product in the form of a cream product that can be applied to the absorbent article or to the skin by hand (for example, as described in detail in U.S. Pat. No. 5,948,416). In another embodiment, the lotion composition can be provided as a stand-alone product in the form of a spray or mousse product that can be sprayed onto the absorbent article or the skin by the wearer of the absorbent article (for example, as described in detail in U.S. Pat. No. 4,708,813).

### Method of Improving Skin Barrier Function of Vulvar Skin

The present invention further encompasses a method of improving skin barrier function of vulvar skin, said method comprising the step of contacting said vulvar skin with an absorbent article comprising a body facing surface and a garment facing surface, wherein omega-6 fatty acid is disposed on said body facing surface of said absorbent article. Improvement in skin barrier function can be exhibited by improved skin lipid composition, improved skin moisturization, or the like.

Generally, a safe and effective amount of the lotion composition is applied to an absorbent article described herein wherein such safe and effective amounts include applying from about 0.05 g/m² (0.032 mg/in²) to about 100 g/m² (64.5 mg/in²) preferably from about 0.5 g/m² (0.32 mg/in²) to about 50 g/m² (32.2 mg/in²), more preferably 1.0 g/m² (0.645 mg/in²) to about 30 g/m² (19.3 mg/in²) of the lotion composition to the absorbent article.

Typically, a safe and effective amount of the lotion compositions of the present invention is applied to an absorbent article such that at least about 0.0005 g/m² (0.00032 mg/in²) to about 50 g/m² (32.3 mg/in²), preferably from about 0.0025 g/m² (0.0016 mg/in²) to about 40 g/m² (25.8 mg/in²), more preferably from about 0.0035 g/m² (0.0022 mg/in²) to about 25 g/m² (16.1 mg/in²), of the composition is transferred to the skin during a single use of an absorbent article which is typically about a three hour period. Absorbent articles are generally changed every three to ten hours during the day and once for overnight protection, resulting in at least a safe and effective amount of from about 0.001 g/m² (0.00064 mg/in²) to about 400 g/m² (218 mg/in²), preferably from about 0.0015 g/m² (0.00096 mg/in²) to about 400 g/m² (218 mg/in²), more preferably from about 0.002 g/m² (0.00128 mg/in²) to about 400 g/m² (218 mg/in²), of the lotion composition being administered within a one day interval (24 hour period). However, the transfer of the lotion compositions of the present invention onto a wearer's skin via an absorbent article described herein can occur for one day, several days, weeks, months, or years at appropriate intervals provided that safe and effective amounts of the lotion compositions are administered to deliver the skin treatment benefits described herein.

Any suitable method can be used in determining the amount of a lotion composition described herein that is transferred to the skin of a wearer during use of an absorbent article containing the composition. Examples of specific methods for the calculation of transfer amounts of lotion compositions include gas chromatography and other quantitative analytical procedures that involve the analysis of in vivo skin analog materials. A suitable gas chromatographic procedure is more fully described in WO 99/45973, Donald C. Roe et al, published Sep. 16, 1999.

The present invention further encompasses the use of an absorbent article comprising omega-6 fatty acid, such as those described herein, for improving the skin barrier function of vulvar skin.

The following are non-limiting examples of the present invention.

### Example I

The compositions exemplified hereinbelow in Table 1 are representative of carrier systems of the lotion compositions of the present invention. The carrier systems are generally prepared by combining, by weight, petrolatum and a fatty alcohol such as behenyl alcohol, and then heating the mixture while stirring to a temperature of about 75° C. using a low speed propeller mixer. Next, viscosity or thickening agents, if present, are added to the mixture to shear mix the ingredients into a final carrier system. Suitable viscosity or thickening agents include beheneth-10, fumed silica, bentonite, and steareth-2, wherein the viscosity or thickening agents are used alone or in combination. The ingredients can be shear mixed at 11,000 revolutions per minute (rpm) using an IKA Ultra Turrax Shear Mixer.

Alternatively, when present, the petrolatum, fatty alcohol, and/or viscosity or thickening agent can be combined, heated with stirring at 75° C. to melt the ingredients, and then mixed into a final carrier system using a high speed blade mixer such as the Tokusyu Kika TK Robo Mics which operates at 5,000 rpm.

TABLE 1
Carrier Systems

| | Sample 1 | Sample 2 | Sample 3 | Sample 4 | Sample 5 | Sample 6 |
|---|---|---|---|---|---|---|
| Component | (Wt. %) | (Wt. %) | (Wt. %) | (Wt. %) | (Wt. %) | (Wt. %) |
| Petrolatum¹ | 74.7 | 77.8 | 83.5 | 100 | 85 | 84.1 |
| Behenyl Alcohol² | 16.2 | 8.7 | 12.2 | | 15 | 12.2 |
| Beheneth-10³ | — | 10 | | | | |
| Fumed Silica⁴ | 4.1 | 3.5 | 4.3 | | | 3.7 |
| Polypropylene Glycol⁵ | 5 | | | | | |

Wt. %—weight percent
¹Petrolatum available as Protopet ® 1S from the Sonneborn Corporation
²Behenyl alcohol available as Lanette 22 from the Cognis Corporation
³Beheneth-10 available as Mergital ® B10 from the Cognis Corporation
⁴Fumed silica available as Cabosil ® TS-720 from the Cabot Corporation
⁵Polypropylene glycol MW 4,000 as Pluriol P4000 from BASF

### Examples II-X

The following Examples II-X illustrated hereinbelow in Table 2 are representative of lotion compositions of the present invention that include the carriers identified in Table 1. The lotion compositions are prepared by formulating a premix solution of the zinc oxide skin treatment agent, if present, and adding the zinc oxide premix to a carrier such as those described in Table 1. The omega-6 fatty acid and other skin treatment agents and any optional ingredients such as panthenol and glycerin, or by formulating a skin treatment solution of hexamidine premix and niacinamide skin treatment agents and any optional ingredients is then added to a carrier such as those described in Table 1 to form the lotion composition, wherein the skin treatment solution and carrier are heated while stirring to a temperature of about 75° C. as described above. Optional ingredients may also be added at later stages while the solution is cooling so long as the solution is above the melt point of the carrier composition. All ingredients are included by weight of the lotion composition.

TABLE 2
Lotion Compositions

| | Ex. II | Ex. III | Ex. IV | Ex. V | EX. VI | Ex. VII | EX. VIII | Ex. IX | Ex. X |
|---|---|---|---|---|---|---|---|---|---|
| Component | (Wt %) | (Wt %) | (Wt %) | (Wt %) | (Wt %) | (Wt %) | (Wt %) | (Wt %) | (Wt %) |
| Sample 1 | 80.21 | — | — | — | — | | 82.00 | — | |
| Sample 2 | — | 91.30 | — | — | — | — | — | 90.75 | — |
| Sample 3 | — | — | 85.80 | — | — | — | — | — | |
| Sample 4 | — | — | — | 96.50 | — | — | — | — | — |
| Sample 5 | — | — | — | — | 99.00 | — | — | — | — |
| Sample 6 | — | — | — | — | — | 87.80 | — | — | 91.40 |
| ZnO Premix⁶ | 7.00 | 3.00 | 10.00 | — | — | 10.00 | 15.00 | — | 7.10 |
| Hexamidine Premix⁷ | 0.67 | 0.15 | 0.15 | — | — | — | — | — | — |
| Panthenol⁸ | 0.50 | 0.50 | 0.50 | — | — | — | | — | — |
| Glycerine⁹ | 0.10 | — | 0.50 | — | — | — | — | — | — |
| Niacinamide¹⁰ | 1.00 | 2.00 | — | — | — | — | — | — | — |
| Acidified Niacinamide¹¹ | — | — | 2.00 | — | — | — | — | — | — |
| Chamomile¹² | 0.50 | 0.50 | 0.50 | — | — | 0.50 | — | — | 0.50 |
| Silk¹³ | 0.02 | 0.05 | — | — | — | — | — | 0.20 | — |
| Lipex ® Omega | 10.00 | — | 0.50 | 2.00 | 1.00 | 0.20 | — | 9.00 | — |
| Passiflora¹⁴ | | | | | | | | | |
| Lipex ® Omega 3/6¹⁵ | — | 2.50 | 0.05 | 1.00 | — | 1.00 | — | — | 1.00 |
| High Oleic Canola Oil¹⁶ | — | — | | 0.50 | — | 0.50 | 3.00 | 0.05 | — |

⁶Zinc oxide premix comprising 70% zinc oxide mixture of ULTRAFINE 350 zinc oxide available from the Kobo Incorporation, Arlecel ® P100 available from the Uniqema Incorporation, and Salacos ® 99 available from the Ikeda Incorporation.
⁷Hexamidine premix available from the Kobo Incorporation comprising 33% hexamidine diisethionate from Laboratories Serolobiologiques under the tradename ELASTAB HP100, Arlecel ® P100 available from the Uniqema Incorporation, and Salacos ® 99 available from the Ikeda Incorporation.
⁸Panthenol available as D-panthenol from DSM Nutritional
⁹Glycerine available as Glycerine, USP Kosher ® from the Procter & Gamble Company
¹⁰Niacinamide available from DSM Nutritional
¹¹Acidified niacinamide made by reacting niacinamide with stearic acid
¹²Chamomile available as Phytoconcentrol Chamomile from Symrise
¹³Silk Protein CROSILK ® from Croda, Inc., of Parsippany, NJ
¹⁴A mixture of vegetable oil and passionflower seed oil commercially-available from Aarhus Karlshamn
¹⁵A mixture of vegetable oil and camelina sativa seed oil commercially-available from Aarhus Karlshamn
¹⁶Available as Clear Valley ® 75 High Oleic Canola Oil from Cargill

The lotion composition of Example II is subsequently applied to the entire wearer-contacting surface of a DRI-WEAVE topsheet of a sanitary pad. To deliver a safe and effective amount of the lotion composition onto the skin, about 4.0 g/m² (2.6 mg/in²) of the lotion composition is applied to the topsheet using a Meltex EP45 hot melt applicator having a head operating temperature of about 90° C.

The lotion composition of Example III is subsequently applied onto the wearer-contacting surface of a hydrophilic spunbond bicomponent polyethylene/polypropylene topsheet (BBA, Washougal, Wash.) of a panty liner product. To deliver a safe and effective amount of the lotion composition onto the skin, the lotion composition is applied to the topsheet in a pattern of circles of at least about 5 mm in diameter and having about 12 g/m² (7.7 mg/in²) of the composition applied thereon. The lotion composition is applied to the topsheet using a hot melt pneumatic Dynatec E84B1758 spray head having a head operating temperature of about 90° C. and an atomization pressure of about 16 kiloPascals (kPa).

The lotion composition of Example IV is subsequently applied by slot coating (Nordsen EP 11-12-02) striped configurations of the composition onto the wearer-contacting surface of a hydrophobic spunbond bicomponent polyethylene/polypropylene topsheet (BBA, Washougal, Wash.) of a sanitary pad product. To deliver a safe and effective amount of the lotion composition onto the skin, the lotion composition is applied to the topsheet in a striped configuration wherein the striped configuration comprises at least six stripes each being 5 millimeters (mm) wide×160 mm long and having about 5.0 g/m² (3.2 mg/in²) of the composition applied thereon.

The lotion composition of Example V is subsequently applied by slot coating (Nordsen EP 11-12-02) striped configurations of the composition onto the wearer-contacting surface of a hydrophobic spunbond bicomponent polyethylene/polypropylene topsheet (BBA, Washougal, Wash.) of a sanitary pad product. To deliver a safe and effective amount of the lotion composition onto the skin, the lotion composition is applied to the topsheet in a striped configuration wherein the striped configuration comprises at least two stripes each being 10 millimeters (mm) wide×at least 120 mm long and at least three stripes each being 5 millimeters (mm) wide×at least 120 mm long having about 15.0 g/m² (4.8 mg/in²) of the composition applied thereon.

The lotion composition of Example VI is subsequently applied by spraying striped configurations of the composition onto the wearer-contacting surface of a DRI-WEAVE topsheet of a panty liner product. To deliver a safe and effective amount of the lotion composition onto the skin, the lotion composition is applied to the topsheet in a striped configuration wherein the striped configuration comprises at least two stripes each being 4 millimeters (mm) wide×at least 40 mm long and having about 1.0 mg/cm² (0.65 mg/in²) of the composition applied thereon. The lotion composition is applied to the topsheet using a hot melt pneumatic Dynatec E84B1758 spray head having a head operating temperature of about 90° C. and an atomization pressure of about 16 kiloPascals (kPa).

The lotion composition of Example VII is subsequently applied by spraying the composition onto the hydrophobic spunbond bicomponent polyethylene/polypropylene topsheet (BBA, Washougal, Wash.) of a sanitary pad product such as Naturella manufactured by the Procter & Gamble Company. To deliver a safe and effective amount of the lotion composition onto the skin, the lotion composition is applied to the topsheet in a pattern of dots of at least about 2 mm in diameter and having about 0.3 g/m² (0.194 mg/in²) of the composition applied thereon. The lotion composition is applied to the topsheet using a hot melt pneumatic Dynatec E84B1758 spray head having a head operating temperature of about 90° C. and an atomization pressure of about 16 kiloPascals (kPa).

The lotion composition of Example VIII is subsequently applied to the entire wearer-contacting surface of a DRI-WEAVE topsheet of a panty liner product. To deliver a safe and effective amount of the lotion composition onto the skin, about 0.5 g/m² (0.32 mg/in²) of the lotion composition is applied to the topsheet using a Meltex EP45 hot melt applicator (or currently supplied by Nordsen) having a head operating temperature of about 90° C.

The lotion composition of Example IX is subsequently applied to the wearer-contacting surface of a DRI-WEAVE topsheet of a sanitary pad. To deliver a safe and effective amount of the lotion composition onto the skin, the lotion composition is applied to the topsheet in a striped configuration wherein the striped configuration comprises at least two stripes each being 20 mm wide×100 mm long and having about 40 g/m² (25.8 mg/in²) of the composition applied thereon. The lotion composition is applied to the topsheet using a hot melt pneumatic Dynatec E84B1758 spray head having a head operating temperature of about 90° C. and an atomization pressure of about 16 kiloPascals (kPa).

The lotion composition of Example X is subsequently applied by slot coating (Nordsen EP 11-12-02) striped configurations of the composition onto the wearer-contacting surface of a hydrophilic spunbond bicomponent polyethylene/polypropylene topsheet (BBA, Washougal, Wash.) of a panty liner product. To deliver a safe and effective amount of the lotion composition onto the skin, the lotion composition is applied to the topsheet in a striped configuration wherein the striped configuration comprises at least four stripes each being 3 millimeters (mm) wide×120 mm long and having about 60 g/m² (38.7 mg/in²) of the composition applied thereon.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of improving skin barrier function of vulvar skin, said method comprising the step of contacting said vulvar skin with an absorbent article comprising a body facing surface and a garment facing surface, wherein omega-6 fatty acid is disposed on said body facing surface of said absorbent article.

2. The method of claim 1, wherein a lotion composition comprises (a) an oil material comprising said omega-6 fatty acid and (b) a carrier, wherein said lotion composition is disposed on said body facing surface of said absorbent article.

3. The method of claim 1, wherein a lotion composition comprises from about 0.00015% to about 10%, by weight of said lotion composition, of said omega-6 fatty acid, and from about 0.0005% to about 16%, by weight of said lotion composition, of oleic acid.

4. The method of claim 2, wherein said oil material is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof.

5. The method of claim 2, wherein said oil material comprises at least about 3%, by weight of the oil material, of omega-6 fatty acid, and wherein said oil material has an oil stability index of at least about 10 hours.

6. The method of claim 2, wherein said lotion composition further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin, and mixtures thereof.

7. The method of claim 3, wherein said lotion composition comprises from about 0.0015% to about 5%, by weight of said lotion composition, of said omega-6 fatty acid.

8. The method of claim 2, wherein said lotion composition further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

9. The method of claim 2, wherein said carrier is selected from the group consisting of petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin, lanolin derivatives, glycerides, glyceride derivatives including acetoglycerides and ethoxylated glycerides of C₁₂-C₂₈ fatty acids, and mixtures thereof.

10. An absorbent article comprising:
a topsheet;
a backsheet;
an absorbent core disposed between said topsheet and said backsheet; and
a lotion composition disposed on one or more layers of said absorbent article, said lotion composition comprising (a) an oil material comprising at least about 3%, by weight of said oil material, of omega-6 fatty acid, wherein said oil material has an oil stability index of at least about 10 hours and is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof, is, and (b) a carrier.

11. The absorbent article of claim 10, wherein said oil material further comprises at least about 10%, by weight of said oil material, of oleic acid, and wherein said oil material has an oil stability index value of at least about 14 hours.

12. The absorbent article of claim 10, wherein said lotion composition further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin, and mixtures thereof.

13. The absorbent article of claim 10, wherein said lotion composition further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

14. The absorbent article of claim 10, wherein said carrier is selected from the group consisting of petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin, lanolin derivatives, glycerides, glyceride derivatives including acetoglycerides and ethoxylated glycerides of C₁₂-C₂₈ fatty acids, and mixtures thereof.

15. An absorbent article comprising:
a topsheet;
a backsheet;
an absorbent core disposed between said topsheet and said backsheet; and
a lotion composition disposed on one or more layers of said absorbent article, said lotion composition comprising: (a) at least about 0.00015%, by weight of said lotion composition, of omega-6 fatty acid, (b) at least about 0.0005%, by weight of said lotion composition, of oleic acid, and (c) a carrier.

16. The absorbent article of claim 15, wherein said oil material is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof.

17. The absorbent article of claim 15, wherein said lotion composition further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin and mixtures thereof.

18. The absorbent article of claim 15, wherein said lotion composition further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

19. The absorbent article of claim 15, wherein said carrier is selected from the group consisting of petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin, lanolin derivatives, glycerides, glyceride derivatives including acetoglycerides and ethoxylated glycerides of C₁₂-C₂₈ fatty acids, and mixtures thereof.

20. A method of improving skin barrier function, lipid composition, or moisturization of vulvar skin, said method comprising the step of contacting said vulvar skin with an absorbent article comprising a body facing surface and a garment facing surface, wherein the lotion composition of claim 3 is disposed on said body facing surface of said absorbent article.
